(19) United States
(12) Patent Application Publication
(10) Pub. No.: US 2018/0125772 A1
(43) Pub. Date: May 10, 2018

(54) Oral Care Compositions

(71) Applicant: Colgate-Palmolive Company, New York, NY (US)

(72) Inventor: Carl Myers, Wayne, NJ (US)

(73) Assignee: Colgate-Palmolive Company, New York, NY (US)

(21) Appl. No.: 15/806,400

(22) Filed: Nov. 8, 2017

Related U.S. Application Data
(60) Provisional application No. 62/419,385, filed on Nov. 8, 2016.

(51) Int. Cl. A61K 8/81; A61K 8/86; A61K 8/49; A61K 8/44; A61Q 11/00; A61Q 19/10

(52) U.S. Cl. CPC A61K 8/8164; A61K 8/86; A61K 8/4926; A61K 2800/594; A61Q 11/00; A61Q 19/10; A61K 2800/48; A61K 8/44

(57) ABSTRACT

This application provides, among other things, novel aqueous biphasic compositions comprising two distinct aqueous phases, useful for combining and delivering poorly compatible ingredients, for example to deliver effective levels of cationic antibacterial agents in combination with anionic polymers, e.g. that protect against erosion and staining, by addition of a stabilizing amount of a polyamine, e.g. lysine, and methods for making and using the same.

### BACKGROUND

This application relates, inter alia, to novel aqueous biphasic compositions useful for combining and delivering poorly compatible ingredients, for example to deliver effective levels of cationic antibacterial agents in combination with polymers that protect against erosion and staining.

Biofilms form when bacteria adhere to surfaces in some form of watery environment and begin to excrete a slimy, glue-like substance that can stick to all kinds of materials—metals, plastics, soil particles, medical implant materials, biological tissues. Dental plaque is a biofilm that adheres to tooth and other oral surfaces, particularly at the gingival margin, and is implicated in the occurrence of gingivitis, periodontitis, caries and other forms of periodontal disease. Dental plaque is cohesive and highly resistant to removal from teeth and/or oral surfaces. Bacteria associated with dental plaque convert sugar to glucans, which are insoluble polysaccharides that provide plaque with its cohesive properties. Anaerobic bacteria in plaque metabolize sugar to produce acids that dissolve tooth minerals, damaging the enamel and eventually forming dental caries. Saliva can buffer acids produced by bacteria and promote remineralization of the enamel, but extensive plaque can block the saliva from contact with the enamel. Redeposition of minerals in the biofilm forms a hard deposit on the tooth called calculus (or tartar), which becomes a local irritant for the gums, causing gingivitis.

Various antibacterial agents can retard the growth of bacteria and thus reduce the formation of biofilm on oral surfaces. In many cases, these antibacterial agents are cationic, for example quaternary ammonium surfactants such as cetyl pyridinium chloride (CPC), bisguanides such as chlorhexidine, metal cations such as zinc or stannous ions, and guanidines such as arginine.

Everyday activities such as smoking or other oral use of tobacco products, and eating, chewing or drinking certain foods and beverages (particularly coffee, tea, cola drinks, and red wine), cause undesirable staining of surfaces of teeth. Staining can also result from microbial activity, including that associated with dental plaque. The chromogens or color causing substances in these materials become part of the pellicle layer and can permeate the enamel layer. Even with regular brushing and flossing, years of chromogen accumulation can impart noticeable tooth discoloration.

A tooth is comprised of an inner dentin layer and an outer hard enamel layer that is the protective layer of the tooth. The enamel layer of a tooth is naturally opaque, and white or a slightly off-white color. The enamel layer is composed of hydroxyapatite mineral crystals that create a somewhat porous surface. These hydroxyapatite crystals form microscopic hexagonal rods or prisms that make up the enamel surface. As a result, the surface of the enamel presents microscopic spaces or pores between the prisms. Without limiting the mechanism, function, or utility of the present disclosure, it is believed that this porous nature of the enamel is where discoloring substances permeate the enamel and discolor the teeth.

As the compounds that stain the teeth are typically anionic materials, cationic antibacterial agents can cause or enhance staining by facilitating the deposit of chromogens or by forming salts with minerals.

One approach to reducing staining and erosion as well as reducing biofilm formation is the use of anionic polymers that help coat and protect the enamel, discouraging bacterial attachment and repelling chromogens. These polymers, however, can interact with cationic antimicrobial agents, leading to formulation incompatibilities, particularly in high water formulations, such as mouthwashes, and inhibiting delivery of the antimicrobial agent and/or the polymer. Oral care products comprising such polymers are disclosed, for example, in WO 2015094336 A1, incorporated herein by reference.

The problems of formulation incompatibilities and treatment of stains on hard surfaces extend not only to teeth but also to hard surface cleansers, such as dish liquids, and the problems of incompatibility in formulation is also seen in personal care products, such as skin cleansers, where it may be desirable to deliver antibacterial agents and/or other skin benefit agents, while avoiding alcohols and oils that may damage the skin.

There is thus a need for novel compositions and methods that minimize interactions between incompatible ingredients in a formulation, enhance delivery of active agents, and inhibit staining and/or biofilm formation.

### BRIEF SUMMARY

It is surprisingly found that formulations comprising an aqueous solution of

a) an acidic polymer, e.g., having an isoelectric point of less than pH 5;

b) a nonionic polymer, e.g. a poly(alkylene oxide);

c); a polyamine compound, e.g., having an isoelectric point of at least pH 8.5, e.g., pH 9-10, e.g., lysine in free or salt form; and

d) water, can form an unusual biphasic system, wherein the polyamine compound interacts with the acidic polymer to form one aqueous phase while the nonionic polymer separates to form a second aqueous phase, the two phases thus both being aqueous, yet having different compositions and densities. If the formulations are shaken to mix the phases, the phases will re-separate when the material is at rest. In certain embodiments, a cationic agent, for example a cationic antibacterial agent, may be included in the formulation, which will be concentrated in the lower phase. The biphasic character provides interesting aesthetic effects, as the addition of a dye makes the two phases visually distinct, and the formulation moreover provides functional benefits by enabling the combination of agents that would otherwise be incompatible. When the phases are mixed, for example by shaking before use, the anionic polymer and any cationic agent are delivered in microdroplets having a relatively high concentration of the anionic polymer and, where present, the cationic active, thus providing improved delivery and a high local concentration of the active at the site of delivery, compared to a homogenous solution. These formulations differ from conventional biphasic formulations in that both phases are aqueous, rather than one phase being hydrophobic and the other hydrophilic. They also differ from structured compositions such as gels insofar as they separate into phases having different densities, e.g., an upper phase and a lower phase, which can be readily mixed by shaking, and which will then re-separate at rest within a short period.

The degree of separation, the relative proportions of the two layers, and the time needed to form two discrete layers can be tuned by varying the polymer and polyamine levels. The dual phase, with a distinct top and bottom layer, each containing different materials and potentially different actives, or at least different concentrations of active, is useful in a variety of applications, e.g.,

for home care use, e.g. in a hard surface cleanser, for example a dishwashing liquid;

for personal care, for example in a liquid hand soap, body wash, sunscreen, or an oil- and alcohol-free skin cleanser; or

for oral care, for example in a mouth wash or other rinse product.

In certain embodiments, the invention provides compositions that form a biphasic aqueous solution using only water soluble materials and no oil. In other embodiments, the aqueous biphasic composition is further combined with an oil phase to give a three-phase system, wherein two of the phases are aqueous and the third is oil-based. In one example, the top layer is composed primarily of mineral oil, the middle layer primarily of water and nonionic polymer, and the bottom layer primarily of anionic polymer and polyamine. These three phases will mix when agitated, and then the layers will separate at rest. This allows for the delivery of further combinations of otherwise insoluble or incompatible active compounds.

For example, cetyl pyridinium chloride (CPC) is useful as an antibacterial agent, while anionic polymers may be useful to help remove and inhibit staining. These ingredients are generally incompatible because they interact, resulting in reduced efficacy both ingredients or even precipitation of both components. The addition of lysine provides needed stability and competition between the acid functional groups of the polymer, the acid and the amine functional groups of lysine, and the CPC—the result is to free CPC and make it more available for interaction with bacteria. In some embodiments, the addition of glutamic acid further improves CPC availability through additional competition pathways through the carboxylates on glutamic acid. Without lysine (and optionally glutamic acid), a formulation with CPC and anionic polymers may have little better efficacy than a non-CPC containing material, or the media control.

Similarly, chlorhexidine will generally complex with anionic polymers no matter what steps are taken, given their high charge density and entropically driven precipitation reaction. But we have found that chlorhixidine and anionic polymers can be formulated in such a way to prevent precipitation (or to re-dissolve the precipitate) through the inclusion of lysine (Lys), polyethylene glycol (PEG), and low levels of anionic surfactant, such as sodium lauryl sulfate (SLS). Additional non-ionic surfactant, e.g., poloxamer, can be used to supplement portions of SLS.

The disclosure thus provides, in one embodiment, compositions comprising an aqueous solution of

(i) an acidic polymer, for example a polymer having an isoelectric point of less than pH 5, e.g., for example synthetic anionic linear polycarboxylates, such as 1:4 to 4:1 copolymers of maleic anhydride or acid with another polymerizable ethylenically unsaturated monomer, e.g., co-polymers of methyl vinyl ether/maleic anhydride, wherein some or all of the anhydride moieties are hydrolyzed to provide free carboxyl groups;

(ii) a nonionic polymer, for example selected from one or more poly(alkylene oxide) polymers, e.g., selected from polyethylene glycols, polypropylene glycols, poloxamers and mixtures thereof; e g., wherein the nonionic polymer has a molecular weight of at least 3000D, e.g., 6kD to 250kD;

(iii) optionally an effective amount of cationic active agent, in free or orally acceptable salt form, e.g., selected from one or more of quaternary ammonium surfactants pyridinium salts, such as cetyl pyridinium chloride), bisguanides (such as chlorhexidine digiuconate), cationic amino acids (such as arginine, in free or salt form), metal cations (such as zinc, calcium, or stannous ions), or combinations thereof;

(iv) a stabilizing amount of a polyamine, e.g., having an isoelectric point of greater than pH 8.5, e.g., lysine, e.g., which may be added in free or salt form; and

(v) water;

wherein the solution comprises two distinct aqueous phases having different composition and density.

The disclosure further provides methods of using such compositions, for example, cleaning hard surfaces, cleaning skin_{;} or inhibiting dental erosion, staining, and/or biofilm formation.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In a first embodiment, the disclosure provides a composition (Composition 1) comprising an aqueous solution of

an acidic polymer;

a nonionic polymer;

optionally an effective amount of cationic active agent;

a stabilizing amount of a polyamine compound; and

water;

wherein the solution comprises two distinct aqueous phases having different composition and density.

For example, the disclosure provides embodiments of Composition 1 as follows:

1.1 Composition 1 wherein the acid polymer is in linear or branched form or mixtures thereof, having acidic functional groups to provide an isoelectric point of pH 5 or less, and optionally additionally having uncharged spacers or side chains, for example comprising hydrophobic moieties (such as methyl methacrylate monomers or alkane chains), and/or uncharged hydrophilic moieties (such as polyalkylene glycols).

1.2 Composition 1 or 1.1 wherein the acidic polymer is a synthetic anionic linear polycarboxylate.

1.3 Composition 1 or 1.1 wherein the acidic polymer is selected from 1:4 to 4:1 copolymers of maleic anhydride or acid with another polymerizable ethylenically unsaturated monomer, e.g., co-polymers of methyl vinyl ether/maleic anhydride, wherein some or all of the anhydride moieties are hydrolyzed to provide free carboxyl groups.

1.4 Any foregoing composition wherein the acidic polymer comprises 0.01 to 30 weight % synthetic anionic linear polycarboxylate, e.g., 0.1 to 30 weight % synthetic anionic linear polycarboxylate, e.g., 1 to 30 weight % synthetic anionic linear polycarboxylate, e.g., 5 to 30 weight % synthetic anionic linear polycarboxylate, e.g., 10 to 30 weight % synthetic anionic linear polycarboxylate, e.g., 10 to 20 weight % synthetic anionic linear polycarboxylate, e.g., 15 weight % synthetic anionic linear polycarboxylate, e.g., 17 weight % synthetic anionic linear polycarboxylate.

1.5 Any foregoing composition wherein the acidic polymer comprises a copolymer of maleic anhydride and methyl vinyl ether.

1.6 Any foregoing composition wherein the acidic polymer comprises a 1:4 to 4:1 copolymer of methyl vinyl ether/maleic anhydride (optionally fully or partially hydrolyzed following co-polymerization to provide the corresponding acid).

1.7 Any foregoing composition wherein the acidic polymer comprises a 1:4 to 4:1 copolymer of methyl vinyl ether/maleic anhydride (optionally fully or partially hydrolyzed following co-polymerization to provide the corresponding acid) having a molecular weight (M. W.) of about 30,000 to about 1,000,000, e.g. about 300,000 to about 800,000.

1.8 Any foregoing composition wherein the acidic polymer is present in a total amount of 1% to 3%.

1.9 Any foregoing composition wherein the nonionic polymer is selected from one or more poly(alkylene oxide) polymers.

1.10 Any foregoing composition wherein the nonionic polymer is selected from polyethylene glycols, polypropylene glycols, poloxamers, co-polymers of polyethylene glycol and polypropylene glycol, and mixtures thereof.

1.11 Any foregoing composition wherein the nonionic polymer has a molecular weight of at least 3000D, e.g., 6kD to 250kD.

1.12 Any foregoing compositions wherein the nonionic polymer comprises polyethylene glycol of MW 5kDa-35kDa, in an amount of 5% to 8%.

1.13 Any foregoing compositions wherein the nonionic polymer is 5-8% polyethylene glycol having a molecular weight of 5kD to 10kD.

1.14 Any foregoing composition wherein the composition comprises a cationic active agent, e.g., a cationic antimicrobial agent.

1.15 Any foregoing composition wherein the composition comprises a cationic active agent, which is an antimicrobial agent, in an antimicrobially effective concentration.

1.16 Any foregoing composition wherein the composition comprises a cationic active agent selected from one or more of quaternary ammonium surfactants (such as cetyl pyridinium chloride (CPC), benzalkonium chloride, cetyl trimethylammonium bromide or chloride, didecyldimethylammonium chloride, benzethonium chloride), bisguanides (such as chlorhexidine digluconate), cationic amino acids (such as arginine), metal cations (such as zinc, calcium, or stannous ions), or combinations thereof, e.g.

1.16.1. Any foregoing composition wherein the composition is an oral care product, e.g., a mouthwash, and comprises an effective amount of an orally acceptable antimicrobial cationic active agent selected from one or more of quaternary ammonium surfactants (such as cetyl pyridinium chloride (CPC)), bisguanides (such as chlorhexidine digluconate), cationic amino acids (such as arginine), metal cations (such as zinc, calcium, or stannous ions), and combinations thereof; or

1.16.2. Any foregoing composition wherein the composition is a personal or home care product, e.g., a skin or hard surface cleanser, and comprises an effective amount of a cationic active which is an antimicrobial cationic surfactant, selected from antimicrobial quaternary ammonium cations (e.g. benzalkonium chloride, cetyl trimethylammonium bromide or chloride, didecyldimethylammonium chloride, cetylpyridinium chloride, benzethonium chloride) and antimicrobial bisguanides (e.g., chlorhexidine digluconate), and combinations thereof.

1.17 Any foregoing composition wherein the composition comprises a cationic active agent comprising a pyridinium surfactant, e.g., cetyl pyridinium chloride (CPC).

1.18 Any foregoing composition wherein the composition comprises a cationic active agent comprising chlorhexidine.

1.19 Any foregoing composition wherein the composition comprises a cationic active agent comprising arginine.

1.20 Any foregoing composition wherein the composition comprises a cationic active agent comprising zinc ions.

1.21 Any foregoing composition wherein the composition comprises a cationic active agent provided by an orally acceptable salt selected from zinc salts, stannous salts, pyridinium salts, and bisguanide salts.

1.22 Any foregoing composition wherein the composition comprises a cationic active agent provided by a salt selected from cetyl pyridinium chloride and chlorhexidine digluconate.

1.23 Any foregoing composition wherein the composition comprises a cationic active agent provided by a zinc salt, stannous salt or combination thereof.

1.24 Any foregoing composition wherein the effective amount of cationic active agent, in free or salt form, is present and comprises cetyl pyridinium chloride, in an amount of 0.05 to 0.1%, e.g., about 0.075%.

1.25 Any foregoing composition wherein the effective amount of cationic active agent, in free or salt form, is present and comprises chlorhexidine digluconate, in an amount of 0.1 to 0.2%, e.g., about 0.12%.

1.26 Any foregoing composition comprising an antimicrobial phenolic compound, e.g., selected from magnolia extract compounds (e.g. magnolol or honokiol), phenol, cresols (e.g., thymol), halogenated (e.g., chlorinated or brominated) phenols (e.g. hexachlorophene, trichlorophenol, tribromophenol, or pentachlorophenol); or an antimicrobial halogenated di-phenyl compound, triclosan, or triclocarban.

1.27 Any foregoing composition wherein the polyamine compound comprises lysine, in free or salt form.

1.28 Any foregoing composition wherein the stabilizing amount of polyamine compound, is an amount sufficient to substantially interfere with interaction between a cationic active agent and the acidic polymer, e.g. an amount sufficient to inhibit formation of a precipitate or reduction of the efficacy of the cationic active agent.

1.29 Any foregoing composition wherein the composition comprises 1%-5% lysine, in free or salt form.

1.30 Any foregoing composition wherein the polyamine is lysine in free or salt form and the composition further comprises glutamic acid, in free or salt form, wherein the combined amount of lysine and glutamic acid is 1 to 10%; e.g., a combination of lysine and glutamic acid in a weight ratio of lysine:glutamic acid of 3:1 to 5:1, wherein the weight % is calculated on the basis of the weight of the free amino acids.

1.31 Any foregoing composition wherein the composition comprises lysine in the form of the hydrochloride salt.

1.32 Any foregoing composition wherein the composition comprises 2%-4% lysine hydrochloride.

1.33 Any foregoing composition further comprising glutamic acid, in free or salt form,

1.34 Any foregoing composition wherein the polyamine, in free or orally acceptable salt form, is lysine, and the composition further comprises glutamic acid, the lysine and the glutamic acid each being in free or orally acceptable salt form, in a total amount of 1 to 10%.

1.35 Any foregoing composition wherein the polyamine, in free or orally acceptable salt form is lysine, and the composition further comprises glutamic acid, each of the lysine and the glutamic acid being in free or orally acceptable salt form and in a weight ratio of lysine:glutamic acid of 3:1 to 5:1, weight being calculated on the basis of the free amino acid.

1.36 Any foregoing composition wherein the composition comprises taurine, e.g., 0.3-3% taurine.

1.37 Any foregoing composition wherein the composition comprises greater than 50% water.

1.38 Any foregoing composition wherein the composition comprises 70% to 95% water.

1.39 Any foregoing composition wherein the composition comprises one or more of a thickener, a buffer, a humectant, a surfactant, an abrasive, a sweetener, a flavorant, a pigment, a dye, an anti-caries agent, an anti-bacterial agent, a whitening agent, a desensitizing agent, a preservative, or a mixture thereof.

1.40 Any foregoing composition wherein the composition contains a bluing agent, e.g., a blue dye or blue pigment, e.g., capable of imparting color to the composition and/or providing a whiter appearance to a yellow surface, for example the surface of a tooth.

1.41 Any foregoing composition wherein the composition comprises a phosphate buffer.

1.42 Any foregoing composition wherein the composition comprises a buffer wherein the buffer comprises sodium hydroxide.

1.43 Any foregoing composition wherein the composition comprises a humectant.

1.44 Any foregoing composition wherein the composition comprises a humectant, wherein the humectant is a mixture of glycerin, sorbitol, and propylene glycol.

1.45 Any foregoing composition wherein the composition comprises an anionic surfactant.

1.46 Any foregoing composition wherein the composition comprises an anionic surfactant, wherein the anionic surfactant is selected from sodium laureth sulfate and sodium lauryl sulfate.

1.47 Any foregoing composition wherein the composition comprises an abrasive.

1.48 Any foregoing composition wherein the composition comprises an abrasive, wherein the abrasive comprises silica.

1.49 Any foregoing composition wherein the composition comprises a sweetener.

1.50 Any foregoing composition wherein the composition comprises a sweetener, wherein the sweetener is sodium saccharin.

1.51 Any foregoing composition wherein the composition comprises a flavorant.

1.52 Any foregoing composition wherein the composition comprises a dye.

1.53 Any foregoing composition wherein the composition comprises an anti-caries agent.

1.54 Any foregoing composition wherein the composition comprises a fluoride ion source.

1.55 Any foregoing composition wherein the composition comprises a fluoride ion source, wherein the fluoride ion source is stannous fluoride, sodium fluoride, potassium fluoride, sodium monofluorophosphate, sodium fluorosilicate, ammonium fluorosilicate, amine fluoride (e.g., N-octadecyltrimethylendiamine-N, N,N′-tris(2-ethanol)-dihydrofluoride), ammonium fluoride, titanium fluoride, hexafluorosulfate, or a mixture thereof.

1.56 Any foregoing composition wherein the composition comprises a whitening agent.

1.57 Any foregoing composition wherein the composition comprises a whitening agent, wherein the whitening agent is hydrogen peroxide.

1.58 Any foregoing composition wherein the composition comprises a desensitizing agent, a vitamin, a preservative, an enzyme, or a mixture thereof.

1.59 Any foregoing composition wherein each of the anionic polymer, the nonionic polymer, the polyamine, and the cationic active agent (if any) are each orally acceptable, e.g., safe for administration to the oral cavity of a human at relevant concentrations.

1.60 Any foregoing composition wherein the composition is a mouthwash, toothpaste, tooth gel, tooth powder, non-abrasive gel, mousse, foam, mouth spray, lozenge, oral tablet, dental implement, or pet care product.

1.61 Any foregoing composition wherein the composition is a mouthwash, e.g., wherein all ingredients of the composition are orally acceptable, e.g., safe and palatable for administration to the oral cavity of a human at relevant concentrations.

1.62 Any foregoing composition which is biphasic, wherein one phase comprises at least 90% of the orally acceptable acidic polymer, the orally acceptable cationic active agent (where present), and the lysine or polylysine, and the other phase comprises at least 90% of the orally acceptable nonionic polymer.

1.63 Any foregoing composition which comprises less than 5%, e.g., less than 2% of hydrophobic ingredients.

1.64 Any foregoing composition which is essentially oil-free, apart from flavoring agents.

1.65 Any of Composition 1-1.64 further comprising an oil phase, e.g., comprising mineral oil.

1.66 Any foregoing composition having a pH between the isoelectric point of the acidic polymer and the isoelectric point of the polyamine compound.

1.67 Any foregoing composition having a pH of 5.5 to 8.0.

1.68 Any foregoing composition having a pH of 5.5 to 6.5.

1.69 Any foregoing composition further comprising an anionic surfactant.

1.70 Any foregoing composition further comprising sodium lauryl sulfate in an amount of up to 1%.

1.71 Any foregoing composition further comprising sodium lauryl sulfate, e.g., 0.1-1.5%.

1.72 Any foregoing composition which is a mouthwash comprising 0.05-0.1%, e.g., about 0.075% cetyl pyridinium chloride; 0.5-2%, e.g., about 1% glutamic acid; 3-5%, e.g., about 4% lysine, 5-7%, e.g., about 6% polyethylene glycol having molecular weight of 8-12 kDa, e.g. about 10 kDa, and 80-90% water.

1.73 Any foregoing composition wherein the composition has any one or more or all of the following features:

a) the acidic polymer comprises a co-polymer of methyl vinyl ether/maleic anhydride in a total amount of 1% to 5%; e.g., about 2%;

b) the nonionic polymer comprises a combination of (i) polyethylene glycol having an average molecular weight of 5kDa to 35kDa, e.g., PEG 8k or PEG 35k, and (ii) poloxamer 407, in a total amount of 3 to 10%; e.g., 2-5% polyethylene glycol and 0.5-2% poloxamer; the cationic active agent is present in an effective amount, in free or orally acceptable salt form and comprises cetyl pyridinium chloride, in an amount of 0.05 to 0.1%, e.g., about 0.075%;

d) the polyamine, in free or salt form, is lysine; and

e) the water is present in an amount of 70-95%; wherein the composition is a mouthwash, further comprising humectant, e.g., propylene glycol 1%-2.5%, e.g., about 1%, flavoring, sweetener, preservative (e.g. potassium sorbate 0.04%-0.06%), and dye (e.g., Blue Dye #1) wherein all ingredients are orally acceptable, e.g., safe and palatable at relevant concentrations for use in a mouthwash, and wherein all amounts are by weight of the total composition.

1.74 Any foregoing composition wherein, upon resting following shaking to mix the phases, the phases separate at room temperature within 30 minutes, e.g., within 15 minutes.

1.75 Any foregoing composition, other than as specifically designated as an oral care composition, wherein the composition is a skin cleanser or hard surface cleanser.

1.76 Composition 1.81 wherein the composition comprises a skin benefit agent, e.g., an antimicrobial agent, e.g., a cationic antimicrobial agent, a moisturizer, and/or a sunscreen.

1.77 Any foregoing composition, wherein

a) the acidic polymer is a co-polymer of methyl vinyl ether/maleic anhydride, and mixtures thereof, in a total amount of 1 to 12%;

b) the nonionic polymer is selected from polyethylene glycol having a molecular weight of 5kD to 35kD, poloxamer 407 in an amount of 0 to 2%, and combinations thereof, in a total amount of 3 to 10%;

c) the effective amount of orally acceptable cationic active agent, in free or orally acceptable salt form, is present and comprises cetyl pyridinium chloride, in an amount of 0.05 to 0.1%

d) the polyamine compound comprises lysine in free or orally acceptable salt form, in a total amount of 1 to 10%; and

e) the water is present in an amount of 35-95%; wherein the composition optionally further comprises an anionic surfactant and/or glutamic acid; and wherein all amounts are by weight of the total composition.

Further claimed is the use of a polyamine, e.g., lysine, in free or orally acceptable salt form, to stabilize an aqueous biphasic formulation, e.g., according to any of Composition 1, et seq., e.g. comprising an acidic polymer, a nonionic polymer, and optionally an effective amount of a cationic active agent, in free or orally acceptable salt form; for example use in any of the foregoing Compositions 1, et seq.

As used herein, an “oral care composition” refers to a composition for which the intended use can include oral care, oral hygiene, or oral appearance, or for which the intended method of use can comprise administration to the oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

As used herein, “cationic active agent” means an agent which is cationic in aqueous solution at neutral pH and which provides some benefit, e.g. antimicrobial activity. In an oral care formulation, the cationic active agent may provide anti-gingivitis, anticavity and/or antierosion activity to the teeth, gums, or oral cavity. While in aqueous formulation, the agent will generally be in solution, but it may be introduced to the formulation formulated in free or salt form. In certain embodiments, for example in certain oral care formulations, the cationic active agent may be selected from one or more of quaternary ammonium surfactants (such as cetyl pyridinium chloride (CPC)), bisguanides (such as chlorhexidine digluconate), cationic amino acids (such as arginine), metal cations (such as zinc, calcium, or stannous ions), or combinations thereof.

As used herein, “acidic polymer” means a polymer comprising monomers bearing acidic groups, for example carboxy groups, for example selected from one or more of synthetic anionic linear polycarboxylates . The acidic polymer should have a relatively low isoelectric point, e.g., pH 5 or less. The appropriate molecular weight will vary depending on the specific polymer, the degree of crosslinking or branching, and the proportion of acidic functional groups, but in general, the molecular weight is greater than 5000 g/mol. In various embodiments, the acidic polymer could be in a linear or nonlinear (i.e. branched) form or a mixture of linear and branched forms, the backbone or side chains could contain various hydrophobic moieties such as methyl methacrylate monomers, alkane chains, etc., and/or as hydrophilic uncharged moieties such as PEG or PPG, as well as moieties bearing acidic functional groups. Examples of acidic polymers include synthetic anionic linear polycarboxylatesand can be selected from a variety of anionic polymers backbones including vinyl, acrylic, maleic. Carboxylate moieties along the polymer backbone can come from the monomers themselves, such as in the case of acrylic acid, methacrylic acid, or maleic acid, or can be generated from the hydrolysis of the polymer, such as in the case of poly-butyl acrylate. The acidic polymer can be made up of copolymers or homopolymers of acidic functional monomers or mixtures thereof.

As used herein, a “nonionic polymer” is a water soluble polymer which does not form an ionic species at relevant pH, e.g., between pH 3 and 10, for example in certain embodiments selected from one or more poly(alkylene oxide) polymers, e.g., selected from polyethylene glycols (PEG), polypropylene glycols (PPG), poloxamers (block co-polymers of PEG and PPG), random copolymers of PEG and PPG, and mixtures thereof. In some embodiments, the nonionic polymer has a molecular weight of at least 3000D, e.g., 6kDa to 250kDa. The molecular weight may vary depending on the particular type of polymer, the degree of branching, if any, and the concentration used. Experiments with PEG having molecular weight between 6 kDa and 35 kDa, for example, showed that at lower concentrations, e.g., for a 3% concentration in a particular combination with other ingredients, a higher molecular weight material, e.g. 35 kDa, was needed to form the biphasic system, but at formulations having higher levels of PEG, a PEG having a lower molecular weight, e.g., 6 kDa could support a biphasic system. In particular embodiments, the nonionic polymer comprises a mixture of (i) polyethylene glycol (MW 5kDa -35kDa) and (ii) poloxamer (i.e., an ethylene oxide/propylene oxide block copolymer), e.g., poloxamer 407, which is a triblock copolymer consisting of a central hydrophobic block of polypropylene glycol flanked by two hydrophilic blocks of polyethylene glycol, wherein the approximate length of the two PEG blocks is about 101 repeat units while the approximate length of the propylene glycol block is about 56 repeat units, available commercially for example as Plutonic F127 (BASF).

As used herein “polyamine compound” means a molecule having at least two primary or secondary amine groups, for example having an isoelectric point of greater than pH 8.5, for example pH 9-10. Examples of polyamines include ethylene diamine, lysine, or histadine, as well as polymers such as Lupasol P, which is a polyethylenimine. The polymine must be safe for its intended use. Where the composition is an oral care composition, the polymaine must be orally acceptable. The polyamine may be provided in free or acid addition salt form. In certain embodiments the polyamine compound is lysine.

As used herein, “biphasic” refers to stable liquid compositions which contain at least two distinct homogeneous phases, having different densities, such that the phases are separate at rest. The phases may be readily mixed by shaking but will then re-separate over a short period, e.g., less than half an hour. In certain embodiments, the term excludes gels, emulsions, microemulsions, and homogeneous solutions. In certain embodiments, these formulations differ from conventional biphasic formulations in that both phases are aqueous, rather than one phase being hydrophobic and the other hydrophilic.

As used herein, “isoelectric point” is the pH in aqueous solution where the molecule has no net charge. To form the biphasic system, three components are needed, two of which are charged—the polyamine compound, e.g. lysine, and the acidic polymer, e.g., Gantrez. The isoelectric point of lysine, for example, occurs at pH 9.7 due to its two amines and one carboxylic acid (at this point only one amine is positive and the acid is negative). At every other pH, Lys contains some degree of charge whether overall positive (<pH 9.7, both amities are protonated) or negative (>pH 9.7, both amities are depronated—neutral—and the acid group has a negative charge). The acidic polymer, e.g., Gantrez S-97, will only have an isoelectric point at low pH <5 at the point where the carboxylates are all protonated resulting in a net 0 charge. The biphasic system exists between the isoelectric points of the necessary materials.

As used herein, “synthetic anionic linear polycarboxylate” refers to a polymer synthesized by using an olefinically or ethylenically unsaturated carboxylic acid that contains an activated carbon-to-carbon olefinic double bond and at least one carboxyl group. The acid contains an olefinic double bond that readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrilacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other olefinic monomers copolymerizable with such carboxylic monomers include vinyl acetate, vinyl chloride, dimethyl maleate and the like. The synthetic anionic linear polycarboxylate is mainly a hydrocarbon with optional halogen and O-containing substituents and linkages as present in for example ester, ether, and OH groups and includes no phosphorus-containing substituents and/or linkages. The copolymers preferably contain sufficient carboxylic salt groups for water-solubility. The terms “synthetic” and “linear” do not include known thickening or gelling agents comprising carboxymethylcellulose and other derivatives of cellulose and natural gums, nor Carbopols having reduced solubility due to cross-linkages.

In some embodiments, “synthetic anionic linear polycarboxylate” refers to 1:4 to 4:1 copolymers of maleic anhydride or acid with another polymerizable ethylenically unsaturated monomer, e.g., methyl vinyl ether (methoxyethylene), having a molecular weight (M.W.) of about 30,000 to about 2,500,000; for example 1:4 to 4:1, e.g. about 1:1, copolymers of methyl vinyl ether/maleic anhydride, wherein the anhydride is hydrolyzed following co-polymerization to provide the corresponding acid, having a molecular weight (M.W.) of about 30,000 to about 1,000,000, e.g. about 300,000 to about 800,000, e.g., as sold under the trade name GANTREZ®, e.g., GANTREZ® S-97 Pharmaceutical Grade (M.W. ca. 700,000), available from Ashland Specialty Chemicals, Bound Brook, N.J. 08805.

As used herein, a “tartar control agent” refers to a compound or a mixture of compounds that inhibit the formation of tartar, a mixture of calcium phosphates on organic matrices, and/or the deposition of plaque on teeth to form tartar (calculus).

As used herein, “chemical stain” refers to a discoloration of a surface, e.g., a dental surface caused by adsorption or absorption of a colored agent on or into the surface, or caused by chemical reaction of material of the surface (e.g., dental enamel) with a colored or noncolored agent contacting the surface. “Chemical staining” herein means formation and/or development of a chemical stain.

As used herein, “dental surface” refers to a surface of a natural tooth or a hard surface of artificial dentition including a crown, cap, filling, bridge, dental implant and the like. in some embodiments, the dental surface is a natural tooth.

Oral care compositions: In some embodiments the compositions are oral care compositions, in accordance with Composition 1, et seq. for example mouthwashes. Any of the compositions of Composition 1, et seq. is suitable for oral care use, provided the ingredients are orally acceptable. In some embodiments, the mouthwash of Composition 1 comprises an effective amount of an orally acceptable cationic active agent, which is an antimicrobial, antigingivitis, anti-erosion and/or anti-caries agent, e,g. a cationic active agent selected from one or more of quaternary' ammonium surfactants (such as cetyl pyridinium chloride (CPC)), bisguanides (such as chlorhexidine digluconate), cationic amino acids (such as arginine), metal cations (such as zinc, calcium, or stannous ions), or combinations thereof. The orally acceptable cationic active agent may be present in an effective amount, for example an antimicrobial, antigingivitis, anti-erosion and/or anti-caries amount. The precise amount will depend on the particular active agent and the condition to be treated or prevented, but in various embodiments, antimicrobially effective levels of CPC in a mouthwash would include amounts from 0.05 to 0.1%, e.g., about 0.075%; antimicrobially effective levels of chlorhexidine digluconate in a mouthwash would include amounts from 0.1-0.2%, e.g., about 0.12%; anti-erosion or anti-microbial levels of metal cations such as zinc (e.g., zinc citrate or other soluble salt) or stannous (e.g., stannous fluoride and/or stannous chloride) would be on the order of 100-1500 ppm.

The oral care composition used in the present disclosure comprise significant levels of water. Water employed in the preparation of commercial oral compositions should be deionized and free of organic impurities. The amount of water in the compositions includes the free water that is added plus that amount which is introduced with other materials.

Mouthwashes frequently contain significant levels of ethanol, which is often needed to solubilize essential oils and to prevent bacterial contamination. High levels of ethanol may be undesirable, because in addition to the potential for abuse by ingestion, the ethanol may exacerbate conditions like xerostoma. Accordingly, in some embodiments, the oral care compositions of the invention are substantially free of ethanol, e.g., contain less than 1% ethanol.

Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case. In some embodiments, humectants are present at levels of 5% to 30%, e.g., 10% to 20% by weight.

Flavorings for use in the present invention may include extracts or oils from flavorful plants such as peppermint, spearmint, cinnamon, wintergreen, and combinations thereof, cooling agents such as menthol, methyl salicylate, and commercially available products such as OptaCool® from Symrise, as well as sweeteners, which may include polyols (which also function as humectants), saccharin, acesulfame, aspartame, neotame, stevia and sucralose.

Further provided is a method (Method A) for the treatment and/or inhibition of a chemical stain, plaque, and/or tartar on a dental surface, comprising shaking the composition according to any of Composition 1, et seq. to disperse the phases and contacting the dental surface therewith.

Further provided herein is Method A as follows:

A.1 Method A wherein the composition is Composition 1, et seq., e.g., wherein the ingredients are orally acceptable, e.g. wherein the composition is a mouthwash.

A.2 Method A or A.1 wherein the method is for the treatment of a chemical stain, plaque, and/or tartar on the dental surface.

A.3 Method A.2 wherein the method is for the treatment of a chemical stain on the dental surface.

A.4 Method A.2 wherein the method is for the treatment of plaque on the dental surfa.ce.

A.5 Method A.2 wherein the method is for the treatment of tartar on the dental surface.

A.6 Method A or wherein the method is for the inhibition of a chemical stain, plaque, and/or tartar on the dental surface.

A.7 Method A.6 wherein the method is for the inhibition of a chemical stain on the dental surface.

A.8 Method A.6 wherein the method is for the inhibition of plaque on the dental surface.

A.9 Method A.6 wherein the method is for the inhibition of tartar on the dental surface.

A.10 Method A or A.1-A.9 wherein the dental surface is a human tooth.

A.11 Method A or A.1-A.10 wherein the composition is contacted with the dental surface by brushing.

Further provided is a method (Method B) for the treatment and/or inhibition of gum disease comprising shaking the composition according to any of Composition 1, et seq. to disperse the phases and contacting the oral cavity therewith.

Further provided herein is Method B as follows:

B.1 Method B wherein the composition is Composition 1, et seq., e.g., wherein the ingredients are orally acceptable, e.g. wherein the composition is a mouthwash.

B.2 Method B or B.1 wherein the method is for the treatment of gum disease.

B.3 Method B, B.1, or B.2 wherein the gum disease is gingivitis.

B.4 Method B, B.1, or B wherein the gum disease is periodontitis.

B.5 Method B or B.1 wherein the method is for the inhibition of gum disease.

B.6 Method B, B.1, or B.5 wherein the gum disease is gingivitis.

B.7 Method B, B.1, or B.5 wherein the gum disease is periodontitis.

B.8 Method B or B.1-B.7 wherein the oral cavity is a human oral cavity.

B.9 Method B or B.1-B.8 wherein the composition is contacted with the oral cavity by brushing.

Further provided is a method (Method for the treatment and/or inhibition of halitosis comprising shaking the composition according to any of Composition 1, et seq. to disperse the phases and contacting the oral cavity therewith.

Further provided herein is Method C as follows:

C.1 Method C wherein the composition is Composition 1, et seq., e.g., wherein the ingredients are orally acceptable, e.g. wherein the composition is a mouthwash.

C.2 Method C or C.1 wherein the oral cavity is a human oral cavity.

C.3 Method C, C.1, or C.2 wherein the composition is contacted with the oral cavity by brushing.

Further provided is a method (Method D) for inhibiting biofilm formation on a dental surface comprising shaking the composition according to any of Composition 1, et seq. to disperse the phases and contacting the dental surface therewith.

Further provided herein is Method D as follows:

D.1 Method D wherein the composition is Composition 1, et seq., e.g., wherein the ingredients are orally acceptable, e.g. wherein the composition is a mouthwash.

D.2 Method D or D.1 wherein the dental surface is a human tooth.

D.3 Method D, D.1, or D.2 wherein the composition is contacted with the dental surface by brushing.

Further provided is a method (Method E) for treating and/or inhibiting bacteria from sticking together and growing into bigger colonies in an oral cavity comprising shaking the composition according to any of Composition 1, et seq. to disperse the phases and contacting the dental surface therewith and contacting the oral cavity therewith.

Further provided herein is Method E as follows:

E.1 Method F wherein the composition is Composition 1, et seq., e.g., wherein the ingredients are orally acceptable, e.g. wherein the composition is a mouthwash.

E.2 Method E or E.1 wherein the oral cavity is a human oral cavity.

E.3 Method E, E.1, or E.2 wherein the composition is contacted with the oral cavity by brushing.

Further provided are Compositions 1, et seq. for use in any of Methods A-E.

As used herein, “inhibition” refers to reduction of stains that would otherwise form or develop subsequent to the time of the treatment. Such inhibition can range from a small but observable or measurable reduction to complete inhibition of subsequent staining, by comparison with an untreated or placebo-treated dental surface.

Where the dental surface is substantially free of chemical stains, Method A, e.g., A.1-A.11, is effective to inhibit formation and development of new chemical stains, as can occur for example by oral use of tobacco products (including smoking) or by drinking tea, coffee, red wine, or coke, subsequent to treatment according to the method. Where the dental surface already possesses some degree of chemical staining, Method A, e.g., A.1-A.11, is effective to inhibit further development of the existing stain. In some embodiments, the Method A, e.g., A.1-A.11, can remove, partially or completely, an existing chemical stain as well as inhibit subsequent staining.

Personal and home care formulations: In another embodiment, the composition of Composition 1, et seq. is a personal care formulation, for example a cleanser such as a liquid hand soap formulation, body wash, or skin cleanser, or a home care formulation, e.g., a hard. surface cleanser such as a dish soap. resulting in a concentration gradient of actives and to give a novel aesthetic. In one embodiment, for example, the composition is a skin cleanser, e.g., an oil-free and alcohol-free skin cleanser, e.g., an antimicrobial, oil-free and alcohol-free skin cleanser.

Any of the compositions of Composition 1, et seq. are suitable for use as cleansers. Typically, skin cleansers will have higher levels of surfactant compared to compositions for oral care use, e.g., anionic surfactants such as sodium laureth sulfate and/or sodium coceth sulfate, e.g., in some embodiments at levels of 5-30%, and in some cases zwitterionic surfactants such as coamidopropyl betaine (CAP-betaine). Dish liquids may have more powerful surfactants, for example amine oxide surfactants, such as lauryl/myristrylamidopropyl dimethylamine oxide

In some embodiments, the personal care compositions of Composition 1, et seq. comprise an effective amount of a skin benefit agent, e.g., an antimicrobial agent, e.g., a cationic antimicrobial agent, a moisturizer, and/or a sunscreen. In some embodiments, the skin benefit agent is a cationic active agent, which may provide skin protection benefits, e.g., as moisturizers, and/or may be antimicrobial cationic active agents, for example antimicrobial quaternary ammonium cations (e.g. benzalkonium chloride, cetyl trimethylammonium bromide or chloride, didecyldimethylammonium chloride, cetylpyridinium chloride, benzethonium chloride) and antimicrobial bisguanides (e.g., chlorhexidine digluconate), and combinations thereof, or bisguanides (such as chlorhexidine digluconate), cationic amino acids (such as arginine), metal cations (such as zinc, calcium, or stannous ions), or combinations thereof. Safe and antimicrobially effective levels of cationic active agents a skin care formulation may be significantly higher than the orally acceptable levels for a mouthwash, e.g., by a factor of 10 to 20. For example, antimicrobially effective levels of chlorhexidine in a skin cleanser would in some embodiments be 3-6%, e.g., about 4%. In some embodiments, the personal care compositions of Composition 1, et seq. contain a skin benefit agent which is a sunscreen agent, e.g., p-aminobenzoic acid (PABA), octyldimethyl-PABA, phenylbenzimidazole sulfonic acid, 2-ethoxyethyl p-methoxycinnamate, benzophenone-8, benzophenone-3, homomethyl salicylate, meradimate, octocrylene, octinoxate, octisalate, sulisobenzone, triethanolamine salicylate, avobenzone, ecamsule, titanium dioxide, zinc oxide, or in some embodiments a basic sunscreen agent, for example a triazole or triazine sunscreen, e.g., bisoctrizolem, bisoctrizole, bemotrizinol, tris-biphenyl triazine, drometrizole trisiloxane, ethylhexyl triazone, and the like.

Further provided is a method (Method F) for cleaning and/or protecting the skin comprising shaking the composition according to any of Composition 1, et seq. and washing the skin therewith.

Further provided herein is Method F as follows:

F.1. Method F wherein the composition is any of Composition 1, et seq., e.g., wherein the composition is a liquid hand soap, body wash, make-up remover, or topical disinfectant.

F.2. Method F or F.1 wherein the composition contains 5-35% anionic surfactant.

F.3. Method F, F.1 or F.2 wherein the composition contains a cationic active agent.

F.4. Method F.3 wherein the composition contains an effective amount of an antimicrobial cationic active agent.

F.5. Any foregoing Method F, et seq. wherein the composition contains an effective amount of a skin-protective cationic active agent, for example to provide a moisturizing and/or sunscreen benefit, or a sunscreen agent.

Further provided is a method (Method G) for cleaning a hard surface, e.g., a dish, comprising shaking the composition according to any of Composition 1, et seq. and washing the hard surface therewith.

Further provided herein is Method G as follows:

G.1. Method G wherein the composition is any of Composition 1, et seq., e.g., wherein the composition is a dish soap or disinfectant.

G.2. Method G or G.1 wherein the composition contains 5-35% anionic surfactant.

G.3. Method G, G.1. or G.2 wherein the composition contains an antimicrobially effective amount of a cationic active agent.

G.4. Method G.3. wherein the method is for the disinfection or disruption of biofilm on a hard surface.

Further provided are Compositions 1, et seq. for use in any of Methods F or G.

### EXAMPLES

Example 1: Stable two-phase formulation

it was found that formulations having 2.5 wt. % of co-polymers of methyl vinyl ether/maleic anhydride together with 4 wt. % polyethylene glycol having a molecular weight of 10,000 g/mol and 4 wt. % Lysine at 6.3 formed a two-phase system.

What is claimed is:

1. A composition comprising an aqueous solution of
a) a synthetic anionic linear polycarboxylate;
b) a nonionic polymer;
c) optionally an effective amount of cationic active agent;
d) a stabilizing amount of a polyamine compound; and
e) water;
wherein the solution comprises two distinct aqueous phases having different composition and density.

2. The composition of claim 1 wherein the synthetic anionic linear polycarboxylate comprises 1:4 to 4:1 copolymers of maleic anhydride or acid with another polymerizable ethylenically unsaturated monomer, e.g., co-polymers of methyl vinyl ether/maleic anhydride, wherein some or all of the anhydride moieties are hydrolyzed to provide free carboxyl groups.

3. The composition of claim 1 wherein the nonionic polymer is selected from polyethylene glycols, polypropylene glycols, poloxamers, and random polyethylene glycol/polypropylene glycol copolymers, and mixtures thereof.

4. The composition of claim 1 comprising a cationic active agent selected from one or more of quaternary ammonium surfactants, bisguanides, cationic amino acids, metal cations, and combinations thereof.

5. The composition of claim 1 comprising a cationic active agent provided by an orally acceptable salt selected from zinc salts, stannous salts, chlorhexidine digluconate, and cetyl pyridinium chloride,

6. The composition of claim 1 wherein the polyamine is lysine, in free or salt form.

7. The composition of claim 1 wherein the composition comprises 70% to 95% water.

8. The composition of claim 1 wherein the composition further comprises an anionic surfactant.

9. The composition of claim 1 having a pH of 5.5 to 8.0.

10. The composition of claim 1 which comprises less than 5% of hydrophobic ingredients.

11. The composition of claim 1 which is essentially oil-free, apart from flavoring agents.

12. The composition of claim 1 wherein the composition further comprises an oil phase.

13. The composition of claim 1 wherein the composition is an oral care composition, wherein the ingredients are orally acceptable, and wherein the composition comprises one or more of a thickener, a buffer, a humectant, a surfactant, an abrasive, a sweetener, a flavorant, a pigment, a dye, an anti-caries agent, an anti-bacterial agent, a whitening agent, a desensitizing agent, a preservative, or a mixture thereof.

14. The composition of claim 1 wherein the composition is a mouthwash.

15. The composition of claim 14 wherein:
a) the a synthetic anionic linear polycarboxylate comprises a co-polymer of methyl vinyl ether/maleic anhydride in a total amount of 1% to 5%; e.g., about 2%;
b) the nonionic polymer comprises a combination of (i) polyethylene glycol having an average molecular weight of 5kDa to 35kDa, e.g., PEG 8k or PEG 35k, and (ii) poloxamer 407, in a total amount of 3 to 10%; e.g., 2-5% polyethylene glycol and 0.5-2% poloxamer;
the cationic active agent is present in an effective amount, in free or orally acceptable salt form and comprises cetyl pyridinium chloride, in an amount of 0.05 to 0.1%, e.g., about 0.075%;
d) the polyamine, in free or salt form, is lysine; and
e) the water is present in an amount of 70-95%, e.g., about 85-90%;
wherein the composition further comprises humectant, e.g., propylene glycol 1%-2.5%, e.g., about 1%, flavoring, sweetener, preservative (e.g. potassium sorbate 0.04%-0.06%), and dye (e.g., Blue Dye #1);
wherein all ingredients are orally acceptable, e.g., safe and palatable at relevant concentrations for use in a mouthwash; and
wherein all amounts are by weight of the total composition.

16. The composition of claim 1 wherein the composition is a skin cleanser.

17. The composition of claim 1 wherein
a) the a synthetic anionic linear polycarboxylate is a co-polymer of methyl vinyl ether/maleic anhydride, and mixtures thereof, in a total amount of 1 to 12%;
b) the nonionic polymer is selected from polyethylene glycol having a molecular weight of 5kD to 35kD, poloxamer 407 in an amount of 0 to 2%, and combinations thereof, in a total amount of 3 to 10%;
c) the effective amount of orally acceptable cationic active agent, in free or orally acceptable salt form, is present and comprises cetyl pyridinium chloride, in an amount of 0.05 to 0.1%
d) the polyamine compound comprises lysine in free or orally acceptable salt form, in a total amount of 1 to 10%; and
e) the water is present in an amount of 35-95%;
wherein the composition optionally further comprises an anionic surfactant and/or glutamic acid; and
wherein all amounts are by weight of the total composition.
